(19) United States
(12) Patent Application Publication
(10) Pub. No.: US 2016/0038434 A1
(43) Pub. Date: Feb. 11, 2016

(54) DEVICE AND METHOD FOR APPLICATION OF A TRANSDERMAL MEMBRANE

(71) Applicants: Michael Schaller, Redwood City, CA (US); Luke Clauson, Redwood City, CA (US)

(72) Inventors: Michael Schaller, Redwood City, CA (US); Luke Clauson, Redwood City, CA (US)

(73) Assignee: INNOVATIVE DRIVE CORPORATION, Redwood City, CA (US)

(21) Appl. No.: 14/817,023

(22) Filed: Aug. 3, 2015

Related U.S. Application Data
(60) Provisional application No. 62/032,408, filed on Aug. 1, 2014.

(51) Int. Cl. A61K 9/70; A61K 31/4468; A61M 35/00; A61K 31/455

(52) U.S. Cl. CPC A61K 9/703; A61K 31/455; A61K 31/4468; A61M 35/003

(57) ABSTRACT

A device and method for moving a membrane into contact and out of contact with the surface of a skin of a patient. The membrane contains at least one active pharmaceutical ingredient (API) and is configured to release the API to the patient when the membrane is in contact with the skin. The device includes components which control the timing of the movement of the membrane based on intended drug delivery profiles. The device may control the amount of surface area of the membrane contacting the skin or the pressure of the membrane on the skin to additionally adjust the drug delivery profile. The membrane may be on a translating platform which moves to a down position where the membrane is in contact with the skin or to an up position where the membrane is not in contact with the skin.

### CROSS-REFERENCES TO RELATED APPLICATIONS

This application claims priority to U.S. provisional patent application No. 62/032,408, filed on Aug. 1, 2014, the full disclosure of which is incorporated herein by reference.

### FIELD OF THE INVENTION

This disclosure relates generally to transdermal drug delivery devices and particularly to a devices for delivering drugs via a movable transdermal delivery patch.

### DESCRIPTION OF THE RELATED ART

Patches have become an effective method for transdermal drug delivery across a wide range of drugs treating a wide range of clinical conditions. For example, fentanyl patches exist which are used for treating patients with moderate to severe pain. Nicotine patches have been used for decades to assist users from quitting smoking. Birth control patches have been used as a replacement for other forms of birth control drug delivery such as pills. Still other drugs and clinical applications exist. Some advantages that have been cited of transdermal drug delivery over other delivery methods are:
Patches are relatively easy to use
Patches reduce the need for patient compliance
Patches provide a more constant drug release profile
Patches may be configured to release drugs at a certain rate versus time

Based on these clear advantages, patches are widely used today. However, many improvements have been contemplated to improve or better control the drug release dynamics. These include pump based systems and other fluid based drug delivery systems where a fluid contains an active pharmaceutical ingredient (API) allowing the drug to be metered in a more controlled way.

### SUMMARY OF THE INVENTION

In one embodiment a device that moves a membrane from a position in contact with the skin of a patient to a position not in contact with the skin wherein the membrane is configured to release an active pharmaceutical ingredient transdermally, is disclosed. In one embodiment the membrane is a transdermal delivery patch.

In one embodiment a wearable device is disclosed, comprising an active pharmaceutical ingredient carrier, and a moveable structure designed to translate said active pharmaceutical ingredient carrier. Translation of said moveable structure is used to modulate the dose of the pharmaceutical ingredient given to the wearer over time. In one embodiment the active pharmaceutical ingredient carrier is a transdermal drug delivery patch. In one embodiment the moveable structure is actuated electromechanically.

### BRIEF DESCRIPTION OF THE DRAWINGS

The invention has other advantages and features that will be more readily apparent from the following detailed description of the invention and the appended claims, when taken in conjunction with the accompanying drawings, in which:

FIG. 1 shows an isometric view of the device with the cover closed

FIG. 2 shows an isometric view of the device with the cover open and a patch not loaded

FIG. 3 shows an isometric view of the device with the cover open and a patch loaded

FIG. 4 shows an isometric cross-section of the device with the patch up

FIG. 5 shows an isometric cross-section of the device with the patch down

FIG. 6 shows an isometric view of the device with the cover hidden and patch up

FIG. 7 shows an isometric view of the device with the cover hidden and patch down

FIG. 8 shows an isometric view of the device with the cover hidden

FIG. 9 shows an isometric view of the device from the underside with the patch up

FIG. 10 shows an isometric view of the device from the underside with the patch down

FIG. 11 shows a cross-sectional view of an alternate embodiment with a diaphragm.

FIG. 12 shows the device of FIG. 11 with the diaphragm moved to a skin contact shape

FIG. 13 shows a cross-sectional view of an alternate embodiment of the diaphragm device with input buttons.

FIG. 14 shows the device of FIG. 13 with a button depressed.

FIG. 15 shows a cross-sectional view of an alternate embodiment of the device with a conveyer belt tread.

FIG. 16 shows the device of FIG. 15 with the tread rotated.

### DETAILED DESCRIPTION

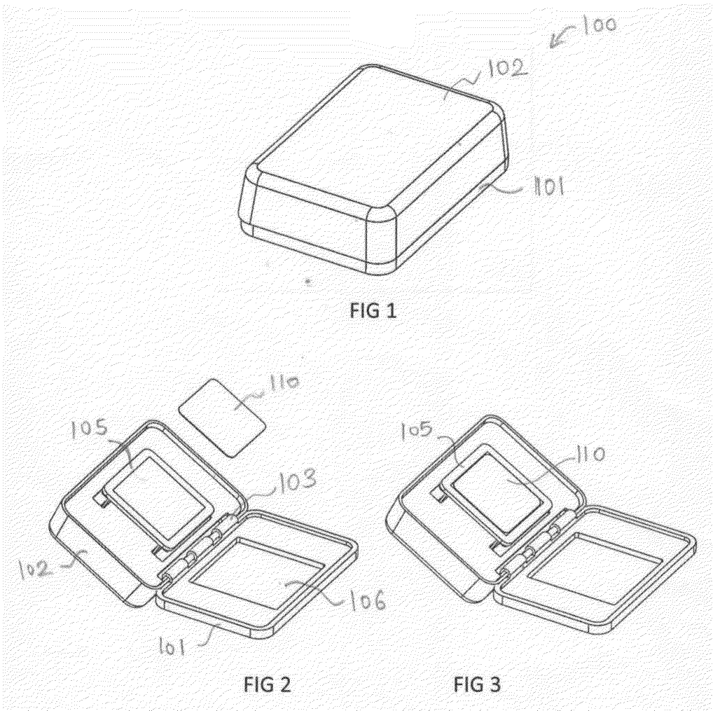

In FIG. 1, one embodiment of the device of the invention is shown. The device 100 includes a base 101 and a cover 102 which may be attached by a hinge or any other suitable method for connecting the components. The base 101 may be affixed to the skin of a patient to provide a surface suitable for transdermal drug delivery. For example, an upper arm, a forearm, an abdomen or any other suitable surface The shape of the base 101 and cover 102 may be a rectangular profile as shown in FIG. 1 or may be any other shape such as a circular or ovular shaped. The base 101 may be affixed to the skin with an adhesive material that is adhered to the undersurface of the base. Alternatively the base 101 may be held against the skin with an arm band, wrist band, or the like that mechanically holds the device against the skin. The bottom surface of the base is shown in the drawings as generally flat, but it should be appreciated that any suitable geometry may be used. For example, if the device is placed on the upper arm of a patient, it may be beneficial for the base 101 to have a curved profile to match the general shape of the bicep. The base 101, and further the whole device 100 may be flexible so as to conform to a variety of different skin shapes and geometries. Additionally, the base 101 may have a permeable mesh or other physical structure to keep the skin from budging into the base hole.

In FIG. 2, the cover 102 of the device is shown opened. A base hole 106 is shown in the base 101 that provides an opening to the patient's skin through the base component. The hole may be shaped and sized appropriately to allow the patch to move through the base hole to contact the patient's skin. A patch mounting plate 105 is shown within the cover assembly and positioned so as to receive a patch 110 placed by a patient onto the patch mounting plate. The patch is shown in FIG. 2 not attached to the device. During normal operation, the patient may open the cover and place the patch onto the patch mounting plate to install a new patch or replace an existing patch with a fresh patch. In FIG. 3, the patch 110 is shown to be placed on the patch mounting plate 105. The patch 110 may be connected to the patch mounting plate 105 with an adhesive that is either on the patch surface of the patch mounting plate or both. The patch mounting plate 105 may have provision for attachment of non-patch drug carrying agents. For example, a tablet of carrier and API that delivers drug when in contact with skin or other physiologic structures could be attached.

In another embodiment (not shown in the figure), a bag with a fluid or suspension including the API could be attached to the patch mounting plate such that the bag is permeable or allows diffusion of API to the target. Many similar API carriers may be contemplated by someone skilled in the art of drug delivery. Alternatively, an additional retaining mechanism may be used to mechanically hold the patch onto the patch mounting plate. For example, a lip or other securement mechanism may exist that goes around the edge of the patch to hold the patch onto the mounting plate. The cover can now be closed onto the base by rotating it about the hinge 103. Any other method of opening and closing the cover onto the hood can be used such as detachable components. For example, the base 101 and or cover 102 may be either a single use disposable or a reusable component. In such an embodiment the manufacturer could supply the cover assembly 102 with the patch already loaded onto the plate and ready for use. Alternatively, the entire device may be supplied ready to use by the supplier.

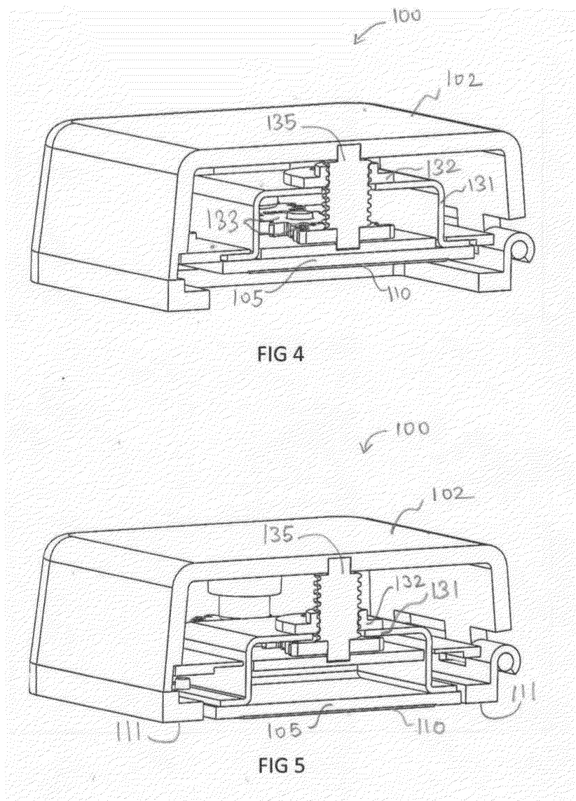

In FIG. 4, a cross-section of the device is shown with the cover 102 closed and a patch 110 loaded. As shown the cover 102 and the base 101 form a relatively sealed compartment that may either be hermetically sealed or just partially sealed. In the inside cavity the patch is shown parallel to the skin contact surface 111. At one state, as shown in FIG. 4, the patch 110 is held in a position above the skin such that it is not contacting the skin. The patch mounting plate 105 may connect to a moving bracket 131. The moving bracket 131 may be connected to a nut 132 which is configured to translate along a lead screw 135 such that when the lead screw 135 rotates the nut 132 moves up and down. A gear exists connected to the lead screw such that as the gears 133 turn the lead screw 135 rotates and the moving bracket 131 moves up and down.

In FIG. 5, the patch is shown in the down configuration such that it is contacting the skin surface. In this configuration the lead screw 135 has been rotated causing the moving bracket 131 and nut 132 to translate along the length of the lead screw. The patch mounting plate 105 and patch 110 therefore move down. The lead screw 105 may be constrained axially by features which exist in the cover 102 and the cover lid which creates the lower surface of the cover assembly. In this manner the lead screw 135 may rotate but not move linearly and has sufficient bearing surface. It should be appreciated that though this embodiment shows linear translation of the patch mounting plate 105 between two end positions the system is in no way limited to linear movement or singular positions.

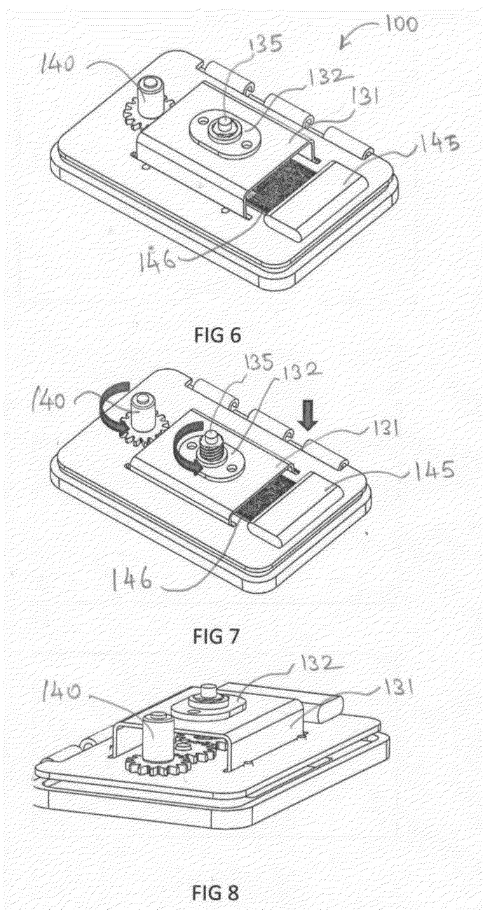

In FIG. 6, an isometric view of one embodiment of the device is shown with the cover hidden to make the inside assembly visible. The moving bracket 131 is shown connected to a nut 132 which is configured to translate along a lead screw 135. A motor 140 exists that is rotationally connected to the lead screw 135 through a set of gears 133. As the motor 140 turns clockwise the patch 110 may move down and contact the skin, and as the motor 140 turns counter clockwise the patch 110 may move up and avoid contact with the skin. The motor 140 may be mounted onto the cover or any other component such that it is capable of rotating the gears with sufficient force. A battery 145 and printed circuit board (PCB) 146 are also shown within the cover assembly. The battery 145 may provide sufficient power for the PCB 146 and for the motor 140. The PCB 146 may include any number of components which are capable of providing electronic triggering for the motor 140 and storing pre-determined move patterns. In addition the PCB 146 may connect with any number of other devices such as smartphones, base stations, host computers or the like. The connected device may send information to the PCB 146 to schedule certain drug delivery profiles and release rates. In such an embodiment the PCB 146 may have a schedule for the drug delivery profiles stored and be capable of moving the patch on and off the skin at the desired times. Alternatively, the connecting device (not shown in the figure) may provide the triggers to the PCB 146 at the desired times such that the PCB 146 does not store pre-determined profiles or events locally. The PCB 146 may connect to the connecting device via BlueTooth, WiFi, or any other suitable method. Further the PCB 146 may have other sensors integrated, such as a gyro, accelerometer, temperature sensor, electrical sensors etc. that take and communicate date to the connected device or are used internally to trigger events.

In FIG. 7, the isometric view is updated to show the patch 110 in the down configuration. As shown, the motor 140 rotates, which rotates the gears 133 and lead screw assembly 135, causing the nut 132 and mounting plate 105 to move down. The patch 110 now contacts the skin and drug may be released transdermally from the patch 110. FIG. 8 shows an alternate isometric view of the device assembly with the cover removed, showing the gears 133 and motor assembly 140. The components within the assembly of the device may be connected via any suitable methods depending on the materials and manufacturing process of the components. For example, the patch mounting plate 105 and moving bracket 131 may be heat staked together or thermally bonded. Alternatively, the components may be press-fit together, connected with mechanical fasteners, or any other suitable method.

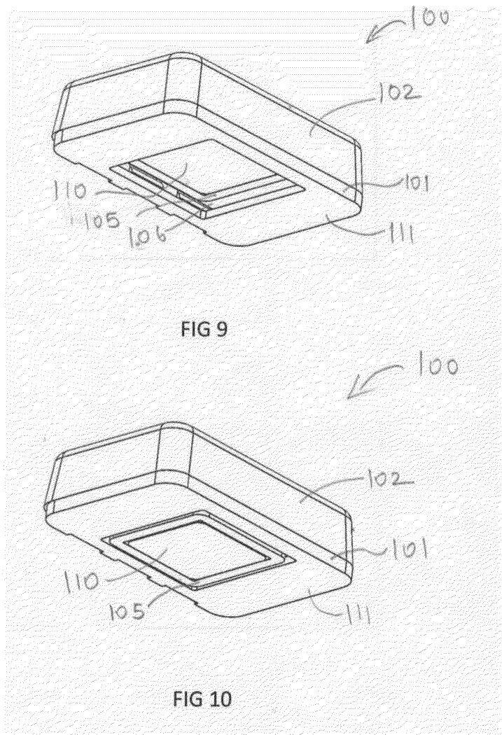

In FIG. 9, the device 100 is shown from an underside view to show the patch in relation to the skin contact surface. In FIG. 9, the patch 110 is shown in the up configuration such that it is not contacting the surface of the skin. The separation gap between the skin and the patch 110 may be between 0.03-0.50 inches or any suitable distance such that the gap is maintained despite a variety of patient skin geometries and fat contents. In FIG. 10, the device is shown with the patch 110 in the down configuration such that the patch 110 contacts the surface of the skin. The patch 110 may be coplanar with the lower surface 111 of the base 101, or alternatively may be lower than the base 101 to provide further pressure onto the skin. The pressure applied may be used to modulate the flow of API in some cases.

Alternatively, instead of moving vertically on and off of the skin, any other number of methods for moving the patch or drug may be considered. For example, the patch may exist on a rotating platform which moves from an angle that is off the surface of the skin and rotates down such that the patch is in intimate contact with the skin. A benefit of such an embodiment may be that the device may contact portions of the skin depending on how far rotated the device is. For example, the device may rotate the patch so that approximately half of the patch is in contact with the skin. This may provide further control for the device to adjust the drug delivery profile and peak dosage delivered.

In still other embodiments the patch or the patch mounting plate may have a geometry such as curved surface that allows the device to adjust the peak drug delivered depending on the pressure supplied to the patch against the skin. For example, if the patch mounting plate is curved, the device may apply a light pressure and only the peak of the curved surface may press into the skin. But if the pressure is curved the skin may contact a wider and wider surface area on the patch.

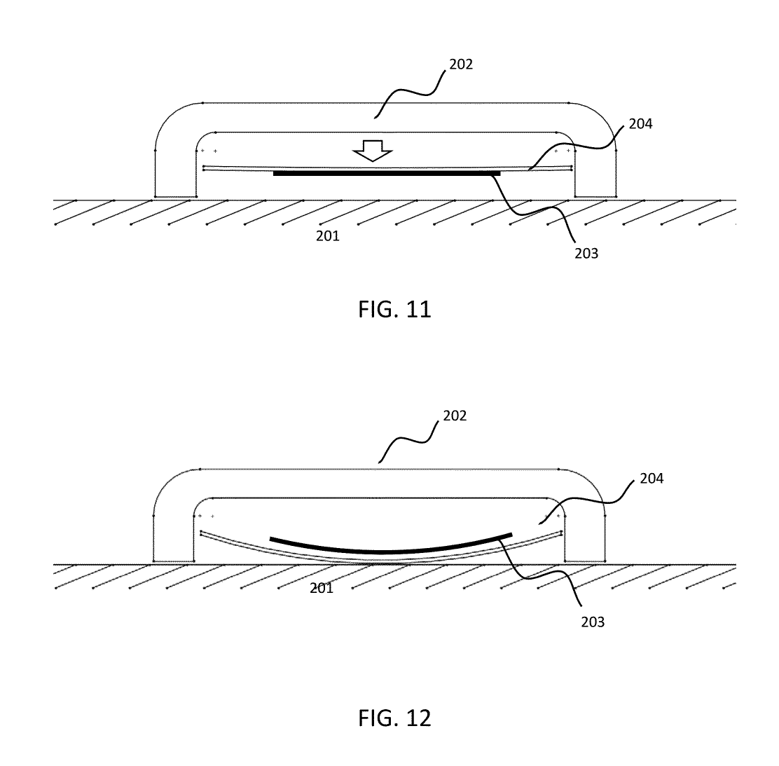

In FIG. 11, an alternate embodiment of the device is shown. The housing 202 of the device rests against the skin 201 of the patient. Contained within the housing 202 exists a membrane 203 attached to a movable diaphragm 204. The diaphragm 204 is shown in a shape that allows the membrane 203 to not be in substantial contact with the skin 201 of the patient. At a predetermined time or based on a desired drug delivery profile, the diaphragm 204 may be configured to change shapes such that the membrane 203 contacts the skin 201. In FIG. 12, the diaphragm 204 is shown extended down and the membrane 203 is contacting the skin 201. Any number of energy sources or mechanical motions may be contemplated for moving the diaphragm 204. In a preferred embodiment, the diaphragm 204 is moved with a pressure source above the diaphragm. The pressure source may be an inert gas such as carbon dioxide which is regulated into the top portion of the housing 202 such that the pressure pushes the diaphragm 204 down. In other embodiments, the diaphragm may be moved with heat activated elements, shape changing elements, mechanically camming elements or any other suitable movement.

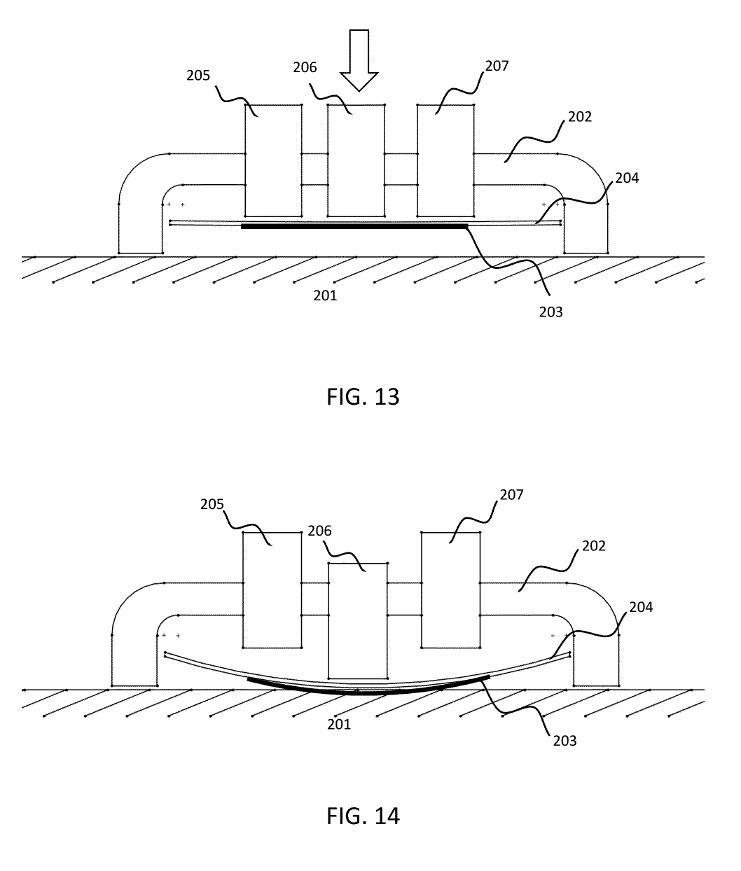

In FIG. 13, an alternate embodiment of the diaphragm device is shown. There are additional buttons along the top of the housing 202. The first button 205, second button 206, and third button 207 may allow the user to mechanically select a desired dosage or delivery rate. The buttons are positioned above the diaphragm 204 such that when the buttons are depressed they move the diaphragm 204 down. In FIG. 14, the device is shown with the middle button 206 depressed. The diaphragm 204 is consequently moved down and presses the membrane into the skin of the patient. As can be seen from the figure, if the first button 205 or third button 207 were also depressed, the surface area of the membrane contacting the patient would be increased. In this way the drug delivery rate or dosage may be adjusted. For example, if the membrane contained fentanyl for treating chronic pain, the patient may be able to select the level of their pain and depress the number of buttons accordingly.

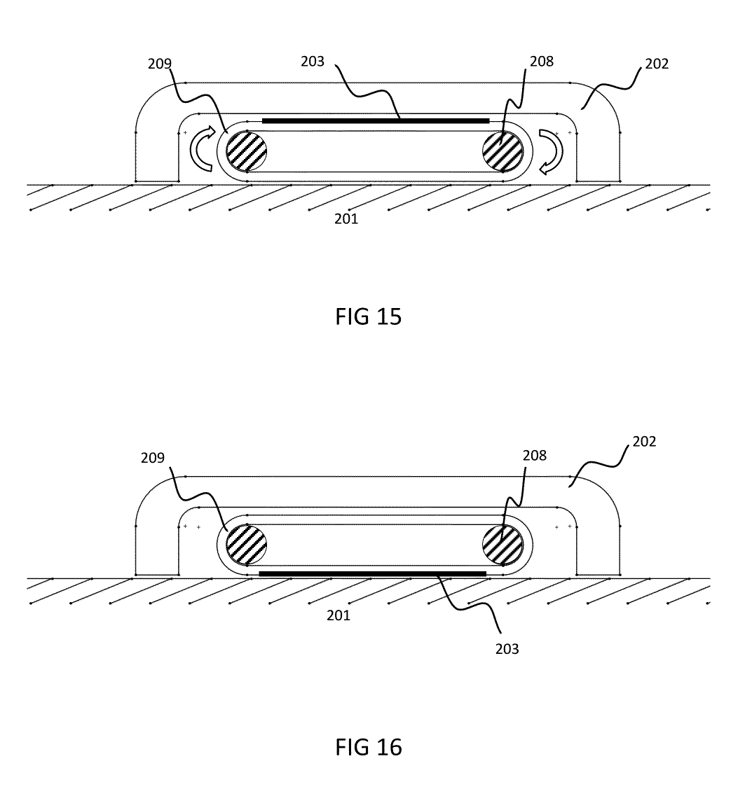

In FIG. 15, an alternate embodiment of the device is shown. The membrane 203 is on a continuous tread 209 which is configured to move like a conveyor belt tread around two pivots 208. The membrane 203 is configured to occupy approximately one whole length of the tread 209 with the opposite length comprised of a material that does not release drug. The tread 209 may spin such that the membrane is either facing the skin 201 or not facing the skin 201. As described above this may allow the device to control the amount of the patch in contact with the skin 201 at any given time, and therefore the drug delivery profile. This method of controlling the drug delivery profile may be beneficial because the patient may use the same membrane 203 and be blinded to the dose they are receiving. In FIG. 16, the device is shown with the tread rotated such that the membrane is in contact with the skin 201 of the patient and administering drug transdermally to the patient.

Additionally, embodiments may be considered where the patch is covered when it is not in contact with the skin. For example, in the tread embodiment described above, the patch may rotate to a position such that the patch is against a surface which covers the patch and prevents the drug from evaporating or otherwise leaving the surface of the patch when it its not in contact with the skin.

In another embodiment, the membrane may not contact the skin of the patient directly, but may still be in communication with the skin. For example, an additional membrane or material may be held against the surface of the skin. This skin membrane may not have any active pharmaceutical ingredients loaded within its structure and instead may act as a cover for the device. In this embodiment, the moving membrane which is loaded with the drug may move into and out of contact with the skin membrane such that when the moving membrane is in contact with the skin membrane it is in communication with the skin of the patient as drug may pass through the skin membrane and transdermally through the patient. It should be appreciated that embodiments herein which describe contact between the membrane and the skin may be interpreted as being in communication with the skin. In still other embodiments, the non-moving skin membrane may be loaded with a certain dose of active pharmaceutical ingredient and the moving membrane may have a different dose of the same or different API. The drug delivery profile may therefore be modulated by moving the moving membrane. In still other embodiments, a stack of several moving membranes may exist such that when they are all in contact and in communication with the skin, the drug delivery rate is related to the number of membranes used. In this way the drug delivery profile may be further modulated by controlling the number of membranes used.

Any number of suitable materials may be considered for the device and individual components. An important consideration is to ensure that the components are generally biocompatible such that no skin irritation or reaction is caused by pressing the components on the skin. In addition, certain components such as the patch mounting plate may need to be compatible with certain drugs or solvents such as fentanyl or nicotine or any other drug or chemical used within the patch. Certain plastics such as polycarbonate, ABS, nylons, polypropylene and the like may be considered for the components such as the base and the cover. Other materials such as the moving bracket may be comprised of metal such as stainless steel and stamped. Gears and lead screws may be made of such metals or of plastic materials. It should be appreciated that any number of materials for any of the components may be considered. In addition, for certain components flexible materials such as thermoplastic elastomers, rubbers and the like may be considered. For example, the patch mounting plate may be comprised of a softer flexible material such that the patch and mounting plate are generally comfortable when pressed against the surface of the skin.

The patch used in such a device may be a commercially available patch such as a Duragesic brand patch or NicoDerm brand patch or any other suitable patch. In such an embodiment the patch may be bought separately by the patient and loaded onto the device, with the device being configured to accept a wide range of patches. The device PCB or connecting device may be configured such that the patient can enter the patch applied to the device. An advantage of such an embodiment is that the commercially available patches are generally well understood and approved by the FDA for transdermal drug delivery. Therefore the amount of clinical research and regulatory filing may be reduced.

Alternatively, a patch specifically designed for the proposed device may be used. For example, a fentanyl or nicotine patch may be created that has no adhesive on the bottom surface of the patch. Instead the patch may be applied to the top surface of the patch such that the patch may be applied to the device. In such an embodiment, the patch and device may be designed so that no other patch may be used with the device with features such as irregular shapes, co-molded plastic components on the plastic, or the like. This may be beneficial such that only a single patch may be used with the device and reduce the likelihood of an incorrect drug delivery profile due to an incompatible device. For example, it may not be ideal for a birth control patch to be used with such a device unless specifically configured for such a use.

As described above the device may be supplied to the patient with the patch already attached to the device. In such embodiments the patch and mounting plate may be one single component. For example, the drug to be released may be stored within any number of components which are configured to move on and off contact with the skin. For example a compliant patch mounting plate may be used and bent into and out of position or a conveyor belt style mounting plate may be employed that exposes portions of the patch or other API carrier to the base hole. It is easily understood how any of these systems could be adapted to vary how much of the API carrier (patch or fluid bag etc.) is exposed to and in contact to with the skin at any time. In still other embodiments, the drug may be stored in a reservoir of fluid which moves. A porous membrane may exist on the surface of the drug reservoir such that the two parts move up and down together against and off of contact with the skin. Alternatively, the membrane may rest against the skin at all times and the drug reservoir may move separately on and off contact with the membrane, either by being translated vertically away or dragged by a conveyor belt, or any other similar method. Still further, the API carrier could have a shield that is moved, thus exposing varying amount of the API's surface. This shield may simply have one hole that is translated, exposing more or less API carrier or it may have a matrix of holes that are of different size or spacing creating various exposure levels of the API carrier to the skin. In this embodiment, translation of the API carrier may or may not be required. Any other number of configurations should be appreciated such that the active substance or drug is moving between a state of contact and non-contact with either the skin or components touching the skin.

In the embodiment as shown in the associated drawings, the patch is configured to move on and off contact with the surface of a patient's skin. In this embodiment, the patient may take the device and open the cover to expose the patch mounting plate. The patient may take a transdermal drug delivery patch which is either supplied with the device or purchased separately and place the patch onto the patch mounting plate. The patient may secure the patch onto the mounting plate with an adhesive or a mechanical method such as a strap or cover or the like. The patient may then close the cover onto the base such that the device is generally closed. The patient may then place the device above a surface of skin where the drug delivery is intended. In a preferred embodiment this may be the upper arm. The patient may secure the device to the upper arm with a strap (not shown in drawings) that mechanically holds the device against the arm. The device may have sensors which determine when the device is in intimate contact with the surface of the skin, and when the patch is loaded, and when the cover is closed, such that the device may know that it is ready to begin any pre-determined drug delivery profile. The device may indicate to the patient that the device is ready for use such as through a visual feedback like an LED or through an audio feedback such as a beep.

When the device determines that a given drug delivery profile should be released, the motor within the device may turn such that the gears and lead screw turn and the nut, moving bracket, and patch move down until the patch makes necessary contact with the skin. The device may adjust the amount of movement to adjust the pressure applied to the skin. For example, for higher doses more pressure may be desired and for lower doses less pressure may be desired. When the drug delivery is desired to stop as determined by the programmed release profile, the motor may turn the opposite direction and translate the patch away from the surface of the skin such that it is no longer in intimate contact with the skin.

The device may be programmed any number of ways. The user may directly input their desired drug delivery profile or discrete delivery times either through the device itself which may include a user input component such as a touch screen, or through a connecting device such as a smartphone which may relay the drug delivery profile. The user may input any amount of drug delivery profiles such as specific release rates and slopes, or times when the most often smoke cigarettes to more closely mimic their typical nicotine blood levels. Additionally, the patient may enter when they experience pain the most and or at what periods of the day they want the side effects such as drowsiness to be minimized The device or connecting device may provide some automated delivery profile information based on the user input. For example, if the patient inputs the times they most often smoke cigarettes, the device or connecting device may adjust the delivery profiles such that the peaks correspond more or less with these times or before or after these times. Alternatively, the device may automatically configure the drug delivery profile for the patient based on limited or no input from the patient. In addition, the device or connecting device may adjust the peak dosage delivered to the patient at a given time or times such that the peak dosage changes over time such as decreases from higher amounts to lower amounts to assist the patient in reducing nicotine dependency. The computational programming or drug delivery profile may be calculated and determined on the device itself through the PCB or through the connecting device and the information relayed to the device. For example the accelerometer may be used to determine when the patient awakes and begin delivery based on such a determination. Alternatively, the device may simply turn on and off in reaction to triggered signals from the connecting device such as a smart phone.

In addition, the device may contain certain user inputs such as a button that allows the patient to select when they need more or less of the drug. For example, if a patient has active pain, a fentanyl may be used within the device to moderate the pain. The device may include buttons or other interfaces which allow the patient to titrate the dose to the amount of pain they have. If they have more pain they can select options on the device to increase the dosage, or alternatively if they have less pain they can select options on the device to decrease the dosage. Further, the device may use the inputs to adjust the drug delivery profile and have algorithms for determining the appropriate adjustment. In the example above, the patient may select their pain level on a scale from 1 to 10 (a common pain scale) and the device may interpret that input and use it for determining the appropriate drug release profile. The device may also understand and have pre-determined limits for safety or other reasons.

After the patient has used the device and a particular patch for a certain amount of time, the drug within the patch or device may diminish and therefore additional patches may need to be reloaded. The patient may open the cover of the device and discard the used patch and place a fresh patch onto the device. In some embodiments, the peak delivery dosage may be determined by controlling the concentration or surface area of the patch.

Although the device embodied in the drawings contains certain elements and configurations, it should be appreciated that any number of other mechanism and devices may be considered.

In addition to the drug delivery embodiments described above, the device may include any number of other sensors and transducers for determining any number of biometric measurements. For example, it may be advantageous to monitor certain biometric variable such as heart rate, blood pressure, body temperature, respiration rate, oxygen saturation, and the like. The data may be used separately of the drug delivery or in conjunction with the drug delivery and provide feedback and adjustment to the delivery profile. The data may be stored either on the device or the connecting device. In one embodiment, the device may determine when the patient has eaten food or woken up or is about to wake up. These events may be useful in determining the correct time to delivery drug.

Further, the device may have sensors or monitoring systems for adjusting the drug delivery profile to an individual patient. For example, the device may include ultrasound to monitor the tissue levels and thicknesses in order to determine the patient's resistance to transdermal drug delivery. Alternatively, the device may also include sensors which are capable of monitoring the real time blood levels of specific drugs or other biometrics such that the drug delivery profile can be tuned to achieve certain biometric goals. For example, the patch may be held onto the surface of the skin until a desired blood level of the drug is achieved. Further this may be used for safety to set threshold limits of maximum applied dosages. Alternatively, the patient may undergo tests at specific time points such as prior to using the device to determine specific metrics or characteristics about the patient. In one embodiment, a patch may be applied for a given amount of time and blood levels may be used to determine actual release rates of the drug across the patient's skin. These measurements may be used as inputs in the device such that the drug release profile may be adjusted to a specific patient.

In still another embodiment, multiple patches may be used and configured to be applied to the skin at the same time or independently different time periods. For example, a patient may have multiple drugs which could be applied transdermally, and the proposed device could accept multiple patches that move independently of one another and create independent drug delivery profiles. In another embodiment, multiple patches of the same drug could be used in a single device such that the patient does not need to remove and reapply a new patch to the device after a single patch has been used. For example, a stack of patches could be stored within the device and applied sequentially to the skin of the patient. Once a single patch has a determined dosage applied, it could be set aside and another patch within the device could be used.

In still other embodiments, the patch could be one continuous band of drug delivery material that is stored across a reel-to-reel mechanism. In this embodiment, a reel of unused drug delivery patch material could be connected to an empty reel across a distance. The space between the reels may be an ideal location for the drug delivery material to contact the skin, perhaps with the aid of a translating component. The device could spin the reels such that a fresh amount of the drug delivery membrane is defined above the skin contact surface and then translated or rotated onto the skin at the pre-determined times. After a given release of drug, the reels may spin further such that the used portion of the material is wrapped onto the empty reel and a fresh unused portion of the material is unreeled into the length above the skin contact area. This may be beneficial because a small device profile may be possible and the user may not be required to change the patch or drug delivery material as often.

In still other embodiments, the device contain washing or flushing mechanism and agents which are applied to the skin or skin contact surfaces to remove any API or drug such that the stoppage of drug release can be controlled closely. This may include streams of water or fluid to spray onto the skin or blotting paper or the like.

Although embodiments of various methods and devices are described herein in detail with reference to certain variation, it should be appreciated that other versions, embodiments, methods of use, and combinations thereof are also possible. Therefore, the scope of the appended claims should not be limited to the description of the embodiments contained herein.

While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope.

What is claimed is:

1. A transdermal drug delivery device that moves a membrane from a first position in communication with a patient to a second position, wherein the first position the membrane has a larger communication area than the second position, and wherein the membrane is configured to release an active pharmaceutical ingredient transdermally when in communication with the skin.

2. The device of claim 1, wherein the membrane contacts the skin directly while in communication.

3. The device of claim 2, wherein the membrane contacts the skin through another medium while in communication.

4. The device of claim 1, wherein the membrane is a transdermal drug delivery patch loaded with the active pharmaceutical ingredient.

5. The device of claim 1, wherein the device is configured to be removably coupled to the body of a patient.

6. The device of claim 1, wherein the membrane has no communication with the skin in the second position.

7. The device of claim 1, wherein the device further comprises a platform attached to the membrane wherein the platform is configured to translate or rotate the membrane between the first and second position.

8. The device of claim 1, wherein the device further comprises a control until which is configured to modulate the timing of the movement to change the transdermal drug delivery profile.

9. The device of claim 8, wherein the control unit modulates the timing of the end of the drug delivery profile.

10. The device of claim 8, wherein the control unit modulates the dosage of drug delivered.

11. The device of claim 1, wherein the active pharmaceutical ingredient is nicotine.

12. The device of claim 1, wherein the active pharmaceutical ingredient is fentanyl.

13. A wearable drug delivery device comprising:
an active pharmaceutical ingredient carrier; and
a moveable structure designed to move said active pharmaceutical ingredient carrier, wherein,
movement of the moveable structure modulates the dose of the active pharmaceutical ingredient given to the wearer over time.

14. The device of claim 13, wherein the active pharmaceutical ingredient carrier is a transdermal drug delivery patch.

15. The device of claim 13, wherein the moveable structure is actuated electromechanically.

16. The device of claim 13, wherein the movable structure is a diaphragm configured to change between a first shape and a second shape.

17. The device of claim 13, wherein the movable structure is a platform configured to translate or rotate between a first position and a second position.

18. A method of transdermal drug delivery comprising:
a carrier structure loaded with at least one active pharmaceutical ingredient and configured to release the active pharmaceutical ingredient to a patient when in contact with the surface of a skin
a movable structure attached to the carrier structure and configured to move between a first position and a second position, wherein the first position the carrier structure has a larger skin contact surface area than the second position
modulating the transdermal drug delivery rate by moving the movable structure between the first and second position

19. The method of claim 18, wherein the timing of the movement between the first position and second position is controlled by a control unit.

20. The method of claim 19, wherein the control unit uses at least one input from the user to adjust the transdermal drug delivery rate.

21. The method of claim 18, wherein the carrier structure is a drug impregnated membrane.
